(19) United States
(12) Patent Application Publication
(10) Pub. No.: US 2024/0358989 A1
(43) Pub. Date: Oct. 31, 2024

(54) FLUID CONNECTOR SYSTEM

(71) Applicant: CareFusion 303, Inc., San Diego, CA (US)

(72) Inventors: Leah Paige Gaffney, Orange, CA (US); Abin Austin, Thrissur (IN); Aman Desai, Bengaluru (IN); Mohammed Mehtab Khan, Bengaluru (IN); Amarsinh Deeliprao Jadhav, Hyderabad (IN); Narsi Reddy Sanikommu, Kanigiri (IN); Anuj Niranjanchavan, Pune (IN); Sachin Gawali, Pune (IN); Prashant Dalavi, Belgaum (IN); Arvind Kumar Mishra, Jhansi (IN); Vinay Kumar Kushwaha, Bihar (IN); Akshay Vidap, Solapur (IN); Richard Edward Byrd, Glen Rock, NJ (US); Nakul Sudarshan, Benguluru (IN)

(21) Appl. No.: 18/306,520

(22) Filed: Apr. 25, 2023

(51) Int. Cl. A61M 39/10

(52) U.S. Cl. CPC A61M 39/1011; A61M 2039/1016; A61M 2039/1066; A61M 2039/1083; A61M 2039/1088

(57) ABSTRACT

Fluid connector systems including first and second connector portions couplable together to form a fluid pathway therethrough and can selectively resist or permit separation as desired. A connector portion can include a connector housing and a cover portion. The connector housing includes a connector body and an engagement portion. The connector body defines a housing volume, a luer opening and a mating opening. The housing volume is in fluid communication with the luer opening and the mating opening. The engagement portion may include an engagement lip extending radially toward the connector body. The engagement lip is configured to releasably engage the connector housing with a mating connector portion. The cover portion is configured to selectively surround the engagement portion of the connector housing. The cover portion is slidable to engage with the engagement portion of the connector housing and prevent release of the connector housing with the mating connector portion.

### BACKGROUND

The present disclosure relates generally to medical fluid connectors and, more particularly, to fluid connector systems having valve assemblies that can be coupled together to form a fluid pathway.

Medical connections are widely used in fluid delivery systems such as those used in connection with intravenous (IV) fluid lines, blood access, hemodialysis, peritoneal dialysis, enteral feeding, drug vial access, and other procedures.

In some instances, the medical connection can become dislodged or disconnected in an unintended manner. For example, medical tubing of an IV set that is coupled to a catheter can become dislodged when an unintended or unexpected forces is exerted upon the catheter, which may exceed the design limitations of the catheter securement method. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

In some applications, high pressure medical fluids (up to or in excess of 325 psi) can be utilized in certain medical infusions. In certain applications, a medical connection can become disconnected when a high-pressure medical fluid is passed through the connection.

### SUMMARY

In accordance with at least some embodiments disclosed herein is the realization that unintended dislodgement or disconnection of a medical connection, such as a medical fluid line, can result in injury to a patient or a caregiver, such as by depriving the patient of a medicament, increasing the potential for infection to the patient, and exposing the caregiver to harmful medicaments.

Accordingly, aspects of the present disclosure provide a connector comprising a first connector portion comprising: a connector housing comprising: a connector body defining a housing volume, a luer opening and a mating opening, wherein the housing volume is in fluid communication with the luer opening and the mating opening; and an engagement portion at least partially surrounding the connector body, the engagement portion defining an engagement lip extending radially toward the connector body, wherein the engagement lip is configured to releasably engage the connector housing with a mating connector portion; and a cover portion configured to selectively surround the engagement portion of the connector housing, wherein the cover portion is slidable to engage with the engagement portion of the connector housing and prevent release of the connector housing with the mating connector portion.

In some instances, the present disclosure provides a connector comprising a first connector portion defining a connector volume, a luer opening and a mating opening, wherein the connector volume is in fluid communication with the luer opening and the mating opening, the first connector portion defining an engagement lip extending radially from the first connector portion, wherein the engagement lip comprises a first lip portion configured to releasably engage an engagement finger of mating connector portion and a second lip portion configured to prevent release of the engagement finger of the mating connector portion.

In some instances, the present disclosure provides a connector comprising a first connector portion defining a connector volume, a luer opening and a mating opening, wherein the connector volume is in fluid communication with the luer opening and the mating opening; a second connector portion comprising defining a second connector volume, a tubing opening and a second mating opening, wherein the second connector volume is in fluid communication with the tubing opening and the second mating opening, wherein the first connector portion and the second connector portion are configured to be separably engageable and the mating opening of the first connector portion and the second mating opening of the second connector portion are in fluid communication when the first connector portion and the second connector portion are in engagement; and an attachable cover portion to selectively surround the first connector portion and the second connector portion and prevent release of the first connector portion and the second connector portion.

Accordingly, the present application addresses several operational challenges encountered in prior fluid connections and provides numerous improvements that enable the user to increase safety and efficacy, while more easily and precisely providing fluid connections.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a fluid connector in use with an IV set coupled to a patient, in accordance with aspects of the present disclosure.

FIG. 2 illustrates a cross-sectional view of a separated fluid connector, in accordance with aspects of the present disclosure.

FIG. 3 illustrates a perspective view of a first connector portion of the fluid connector of FIG. 2, in accordance with aspects of the present disclosure.

FIG. 4A illustrates a connector body of the first connector portion of FIG. 3, in accordance with aspects of the present disclosure.

FIG. 4B illustrates a cover portion of the first connector portion of FIG. 3, in accordance with aspects of the present disclosure.

FIG. 5A illustrates a perspective view of the first connector portion of the fluid connector of FIG. 2, in accordance with aspects of the present disclosure.

FIG. 5B illustrates a perspective view of the first connector portion of the fluid connector of FIG. 2, in accordance with aspects of the present disclosure.

FIG. 6 illustrates a cross-sectional view of a fluid connector, in accordance with aspects of the present disclosure.

FIG. 7A illustrates a cross-sectional view of a fluid connector in a locked configuration, in accordance with aspects of the present disclosure.

FIG. 7B illustrates a cross-sectional view of a fluid connector in an unlocked configuration, in accordance with aspects of the present disclosure.

FIG. 8 illustrates a perspective view of a lock ring of the fluid connector of FIGS. 7A and 7B, in accordance with aspects of the present disclosure.

FIG. 9 illustrates a perspective view of a fluid connector, in accordance with aspects of the present disclosure.

FIG. 10 illustrates a perspective view of a first connector portion, in accordance with aspects of the present disclosure.

FIG. 11 illustrates a cross-sectional view of the first connector portion of FIG. 10, in accordance with aspects of the present disclosure.

FIG. 12 illustrates a perspective view of a second connector portion, in accordance with aspects of the present disclosure.

FIG. 13 illustrates a cross-sectional view of the second connector portion of FIG. 12, in accordance with aspects of the present disclosure.

FIG. 14A illustrates a cross-sectional view of a fluid connector in a locked configuration, in accordance with aspects of the present disclosure.

FIG. 14B illustrates a cross-sectional view of a fluid connector in an unlocked configuration, in accordance with aspects of the present disclosure.

FIG. 15 illustrates a cross-sectional view of a separated fluid connector, in accordance with aspects of the present disclosure.

FIG. 16 illustrates a perspective view of a fluid connector with a cover, in accordance with aspects of the present disclosure.

FIG. 17 illustrates a perspective view of a fluid connector with a cover, in accordance with aspects of the present disclosure.

FIG. 18 illustrates a cross-sectional view of a fluid connector with a cover, in accordance with aspects of the present disclosure.

FIG. 19A illustrates a perspective view of a cover for a fluid connector in a closed configuration, in accordance with aspects of the present disclosure.

FIG. 19B illustrates a perspective view of a cover for a fluid connector in an open configuration, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a fluid connector system. The fluid connector system can provide for efficient and safe maintenance of fluid connections, such as the connections used for transferring medical fluids toward or away from a patient. The fluid connector system can maintain a fluid pathway by resisting unintended disconnection when a pulling or tension force is applied to the fluid connector system, such as when a patient moves or when the medical tubing is pulled away from the patient.

The fluid connector system can also prevent injury to a patient or a caregiver by permitting disconnection or separation between portions of the connector system when a pulling or tension force exceeds a threshold. The fluid connector system can also prevent injury to a patient or a caregiver by obstructing the fluid pathway when disconnection or separation between portions of the connector system occurs. Further, the fluid connector system can provide for efficient and safe reestablishment of the fluid pathway, by permitting reassembly of portions of the system after a disconnection or separation occurs. Advantageously, the fluid connectors described herein can prevent blood loss, IV fluid loss, infection, and/or delays in medication delivery. In some applications, the design of the fluid connectors can facilitate effective cleaning and sanitization of the components.

Further, in some applications, the fluid connector system can selectively resist unintended disconnection during certain events or applications, and selectively but allow for disconnection or separation between portions of the connector system when a pulling or tension force exceeds a threshold. For example, a fluid connector system can be configured to resist disconnection or separation during high pressure infusion (fluid pressures up to or in excess of 325 psi) or during other clinical operations that may cause the fluid connector system to unintentionally separate. The fluid connector system may be reconfigured to selectively allow for disconnection or separation between portions of the connector system when a pulling or tension force exceeds a threshold.

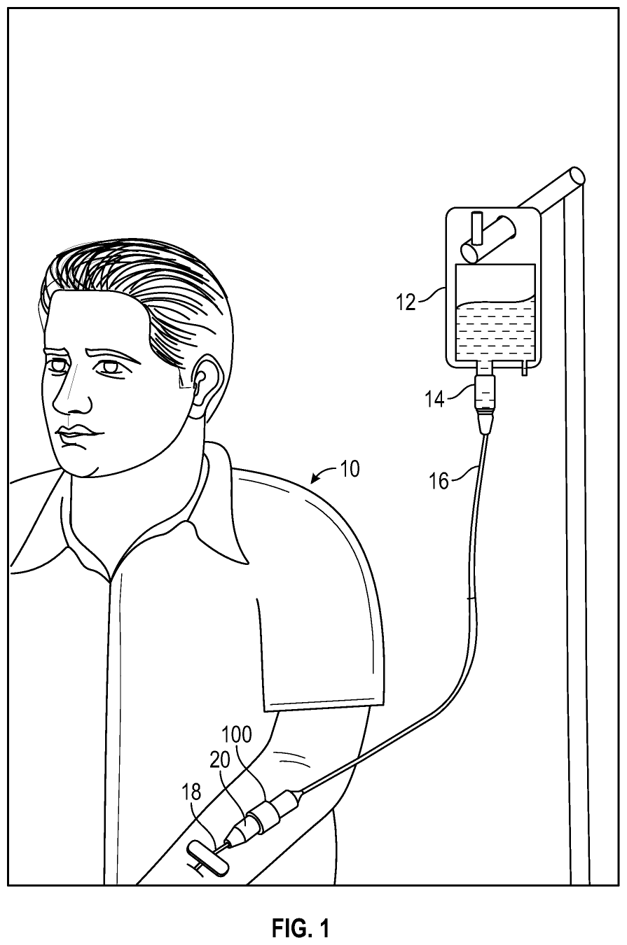

Referring now to the figures, FIG. 1 illustrates an example of a fluid connector 100 in use in accordance with aspects of the present disclosure. The connector system 100 is coupled with tubing of an IV set, which is being used to direct a fluid to a patient 10. The IV set can include a medicament bag 12, a drip chamber 14, tubing 16, and an IV catheter 18.

The connector system 100 fluidly connects the tubing 16 to the IV catheter 18. Although the connector system 100 is illustrated being coupled along a fluid pathway of an IV set. between a medicament bag 12 and a patient 10, it should be understood that the connector system 100 can be connected within other fluid pathways, such as between a patient and an IV pump or between a patient and a dialysis machine. The connector system 100 can also be connected along another portion of a fluid pathway. For example, the connector system 100 can be connected along a proximal portion of the fluid pathway, such as being connected between the tubing 16 and the medicament bag 12 or other fluid therapy device. In another example, any of the first and second portions of the fluid connector 100 can be directly coupled to another fluid delivery devices, such as the catheter or a medicament bag.

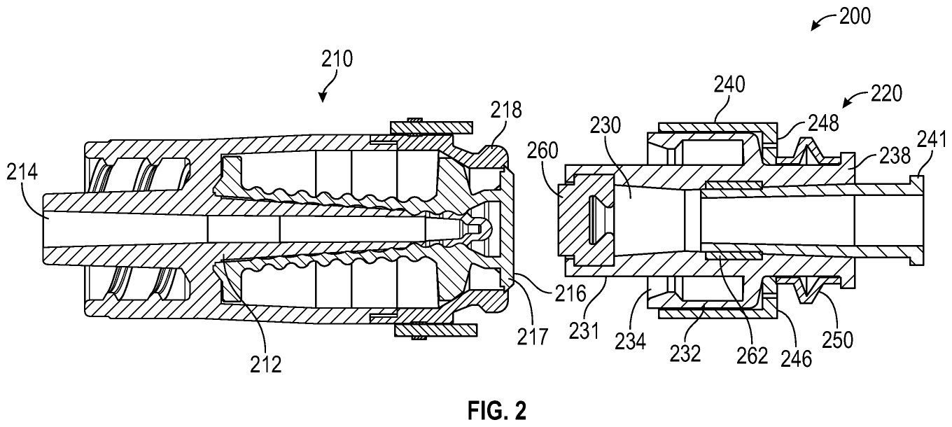

FIG. 2 illustrates a cross-sectional view of a separated fluid connector 200, in accordance with aspects of the present disclosure. With reference to FIG. 2, the fluid connector 200 provides a fluid pathway to a patient while allowing for a selectable “fusible link” or quick disconnect, allowing for a controlled disconnection of the fluid connector 200 if excess force is applied and allowing for a maintained connection during high pressure infusions or other procedures. As described herein, the fluid connector 200 can allow for controlled disconnection at a predetermined level of force to prevent a catheter from being inadvertently removed from the patient or prevent inadvertent disconnection during high pressure infusion procedures.

As illustrated, the fluid connector 200 provides a flow path or fluid pathway from one end of the fluid connector 200 to the opposite end of the fluid connector 200. As illustrated, the fluid connector 200 includes a first connector portion 220 and a second connector portion 210 that are coupled together to form a fluid pathway.

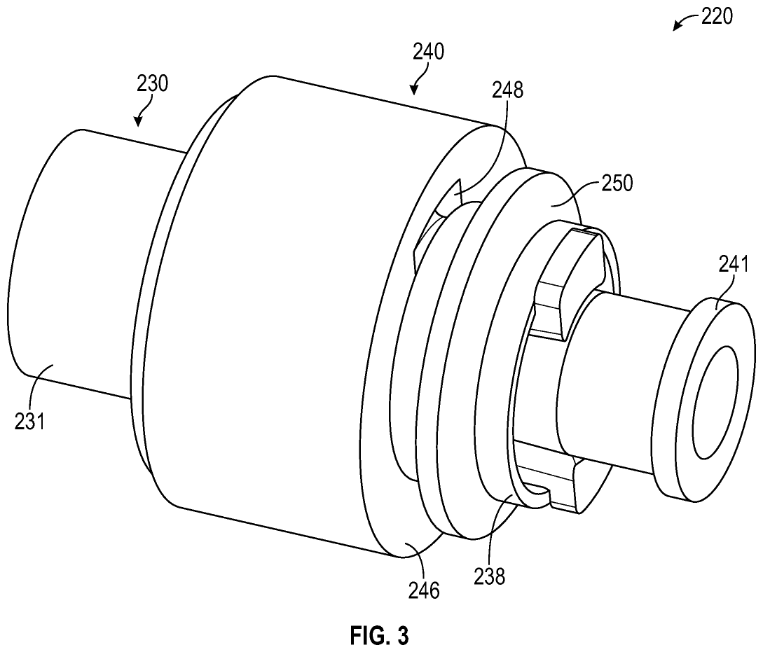

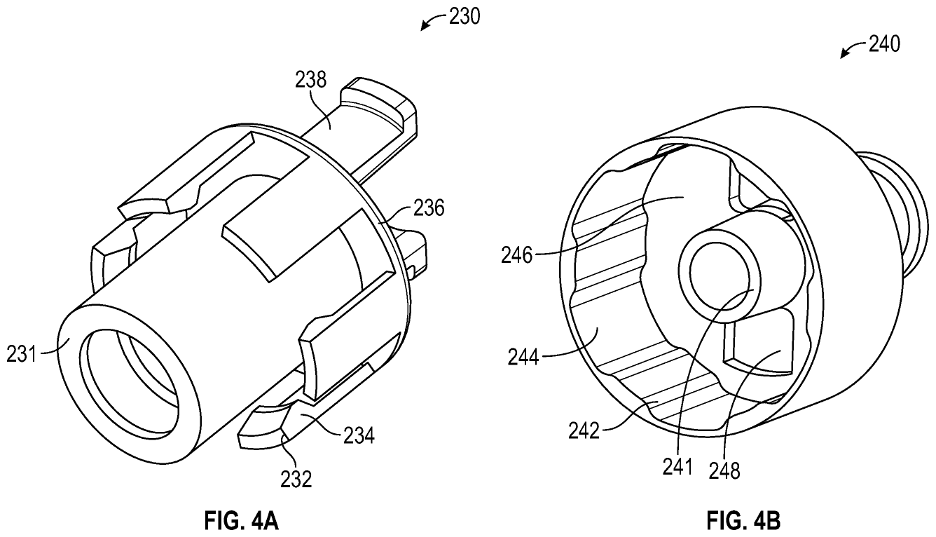

FIG. 3 illustrates a perspective view of a first connector portion 220 of the fluid connector 200 of FIG. 2, in accordance with aspects of the present disclosure. FIG. 4A illustrates a connector body 230 of the first connector portion of FIG. 3, in accordance with aspects of the present disclosure. FIG. 4B illustrates a cover portion 240 of the first connector portion of FIG. 3, in accordance with aspects of the present disclosure. With reference to FIGS. 2-4B, the first connector portion 220 permits fluid flow to and from the patient or any other portion of the IV set. In the depicted example, the first connector portion 220 includes a sliding portion 240 to allow the first connector portion 220 to be connected to mating tubing or luer connector. As illustrated, a mating luer connector can be attached to the lumen 241. Fluid can pass to and from the fluid connector 200 through the mating luer connector via a lumen 241 defined in the sliding portion 240.

In the depicted example, the sliding portion 240 can be coupled to the other portions of the first connector portion 220. In some embodiments, a housing end of the sliding portion 240 can be coupled to a luer end of a connector body 230 allowing the lumen 241 of the sliding portion 240 to be in fluid communication with the lumen 231 of the connector body 230 and generally allowing fluid communication between the sliding portion 240 and the connector body 230. As illustrated a portion of the lumen 241 of the sliding portion 240 can be disposed within the lumen 231 of the connector body 230. Optionally, the first connector portion 220 can include a seal 262 to maintain isolation between the lumen 231 of the connector body 230 and the lumen 241 of the sliding portion 240. As described herein, the sliding portion 240 can slide relative to the connector body 230. In some embodiments, the seal 262 can maintain a seal or isolation between the lumen 231 and the lumen 241 as the sliding portion 240 moves or slides relative to the connector body 230.

As illustrated, the sliding portion 240 mated, coupled, or otherwise retained with the connector body 230. In some embodiments, one or more retaining fingers 238 extend from a base 236 of the connector body 230 toward the sliding portion 240. The retaining fingers 238 can extend through one or more windows 248 formed in the base 246 of the sliding portion 240 and radially expand outward to retain the sliding portion 240 relative to the connector body 230. During operation, the retaining fingers 238 can limit the sliding or axial travel of the sliding portion 240 relative to the connector body 230. In some applications, the retaining fingers 238 can be compressed radially inward to allow the sliding portion 240 to be removed from the connector body 230.

Further, the second connector portion 210 permits fluid flow to and from a fluid source or any other portion of the IV set. In the depicted example, the second connector portion 210 can be connected to a fluid source or other portion of the IV set. Tubing or a connector can be received in an opening 214 of the connector housing and can be in fluid communication with a lumen 212 of the second connector portion 210.

During operation, the connector body 230 of the first connector portion 220 and the second connector portion 210 can be coupled together to permit flow between the first connector portion 220 and the second connector portion 210. In the depicted example, the lumen 231 of the connector body 230 can define a mating opening in fluid communication with a housing volume and the lumen 241 of the sliding portion 240. Similarly, the second connector portion 210 can define a mating opening 217 in fluid connection with a housing volume, the lumen 212, and the tubing or other IV set component coupled to the opening 214 of the second connector portion 210. As the first connector portion 220 and the second connector portion 210 are coupled together, fluid can flow between the mating opening of the first connector portion 220 and the mating opening 217 of the second connector portion 210, allowing flow between the first connector portion 220 and the second connector portion 210.

In the depicted example, the first connector portion 220 and the second connector portion 210 each include a valve member 260, 216 respectively to control flow through the connector 200. In particular, the valve members 260, 216 allow for fluid to flow between the first connector portion 220 and the second connector 210 when the first connector portion 220 and the second connector portion 210 are connected and prevent fluid flow when the first connector portion 220 and the second connector portion 210 are disconnected.

In the depicted example, the first connector portion 220 and the second connector portion 210 can be engaged to secure or retain the first connector portion 220 and the second connector portion 210 together. As illustrated, the first connector portion 220 and the second connector portion 210 can be coupled together by securing a portion of the first connector portion 220 around the second connector portion 210. In the depicted example, the connector body 230 of the first connector portion 220 includes or defines one or more engagement fingers 232. As illustrated, the engagement fingers 232 extends axially away from the base 236 of the connector body 230. Optionally, the engagement fingers 232 can be disposed circumferentially around the lumen 231 of the connector body 230. In some embodiments, the engagement fingers 232 can each define a ridge or lip 234 that extends radially inward toward the lumen 231. Similarly, the second connector portion 210 defines a groove 218. Optionally, the groove 218 can be disposed circumferentially around the lumen 212 of the second connector portion 210. Further the groove 218 can be defined on the outer surface of the second connector portion 210. During operation, the engagement fingers 232 of the first connector portion 220 can engage with the groove 218 of the second connector portion 210 to couple the first connector portion 220 and the second connector portion 210. In some embodiments, the lip 234 of the engagement fingers 232 can engage with the groove 218 of the second connector portion 210.

In the depicted example, the engagement fingers 232 and/or the lip 234 of the first connector portion 220 engages the groove 218 of the second connector portion 210 to resist movement of the first connector portion 220 and the second connector portion 210 in a direction away from each other. Engagement of the engagement fingers 232 against the groove 218 can, in some instances of the present disclosure, define a snap fitting or snap joint between the first connector portion 220 and the second connector portion 210. In some embodiments, the engagement fingers 232 can deflect to facilitate engagement or disengagement of the engagement fingers 232 with the groove 218.

Although the engagement fingers 232 are configured to resist separation of the first connector portion 220 and the second connector portion 210, the engagement fingers 232 and/or groove 218 are also configured to permit separation of the first connector portion 220 and the second connector portion 210 when a threshold force exceeded between the first connector portion 220 and the second connector portion 210. In some embodiments of the present disclosure, the threshold force for separating the first connector portion 220 and the second connector portion 210 is greater than or equal to approximately five pounds (22.25 Newtons). Separation of the first connector portion 220 and the second connector portion 210 can occur when the engagement fingers 232 of the first connector portion 220 are biased or flexed in a direction away from the groove 218. In the depicted example, the first connector portion 220 and the second connector portion 210 can separate from each other while maintaining the ability to reconnect the first connector portion 220 and the second connector portion 210 together. In some embodiments, the resistance or force to assembly and separation between the first connector portion 220 and the second connector portion 210 can be configured so that the force required for assembly of the first connector portion 220 and the second connector portion 210 is less than the force required for separation the first connector portion 220 and the second connector portion 210.

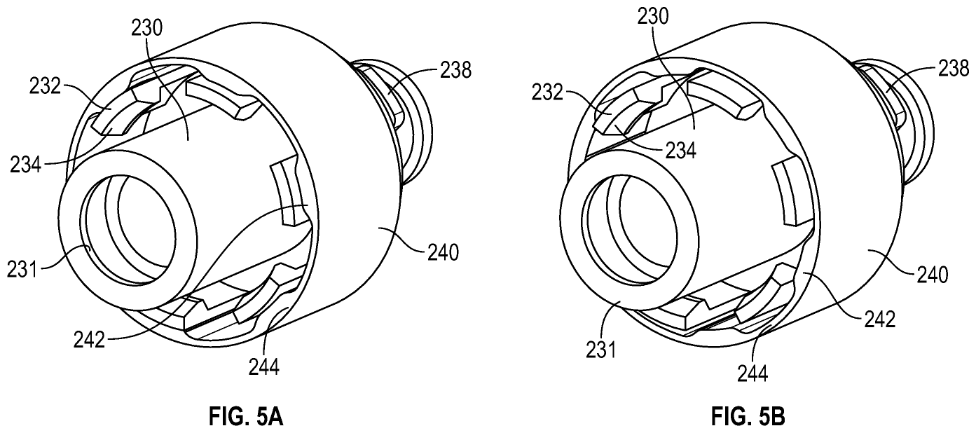

FIG. 5A illustrates a perspective view of the first connector portion 220 of the fluid connector 200 of FIG. 2, in accordance with aspects of the present disclosure. With reference to FIGS. 2-5A, the connector 200 can prevent the unintended disconnection of the first connector portion 220 and the second connector portion 210, during certain applications, such as high-pressure fluid infusions. In the depicted example, the first connector portion 220 can include a sliding cover 244 to selectively prevent the disengagement of the engagement fingers 232 of the first connector portion 220 from the groove 218 of the second connector portion 210.

In a locking position, the sliding cover 244 can be aligned with the engagement fingers 232 to prevent the radial or outward deflection of the engagement fingers 232, thereby preventing the disengagement of the engagement fingers 232 from the groove 218 of the second connector portion 210. In some applications, the sliding cover 244 includes one or more protrusions 242 extending radially toward the engagement fingers 232 to prevent the outward radial movement of the engagement fingers 232.

During operation, the sliding cover 244 can be moved or slid away from the engagement fingers 232 to allow the engagement fingers 232 to radially or outwardly deflect, permitting the engagement or disengagement of the first connector portion 220 from the second connector portion 210. A clinician may apply an external force to translate or move the sliding cover 244 to a desired position. As illustrated, the sliding cover 244 can be formed with the sliding portion 240. In some embodiments, the sliding cover 244 is circumferentially disposed around the lumen 241 of the sliding portion 240. In some embodiments, the first connector portion 220 can include a biasing member 250 to urge the sliding cover 244 toward a locked or retained position. The biasing member 250 can be a bellows type spring. Optionally, the biasing member 250 can be disposed between the base 246 of the sliding cover 244 and the retaining fingers 238 of the connector body 230. In some applications, a clinician may apply an external force to overcome the spring force of the biasing member 250 to move the sliding cover 244 to an unlocked position.

FIG. 5B illustrates a perspective view of the first connector portion 220 of the fluid connector 200 of FIG. 2, in accordance with aspects of the present disclosure. With reference to FIG. 5B, the protrusions 242 of the sliding cover 244 can define slots therebetween which may allow the radial or outward deflection of the engagement fingers 232, permitting the disengagement of the first connector portion 220 from the second connector portion 210. During operation, the sliding cover 244 can be rotated to align the protrusions 242 with the engagement fingers 232, preventing disconnection, or rotated to align the slots between the protrusions 242 with the engagement fingers 232, allowing disconnection of the first connector portion 220 from the second connector portion 210.

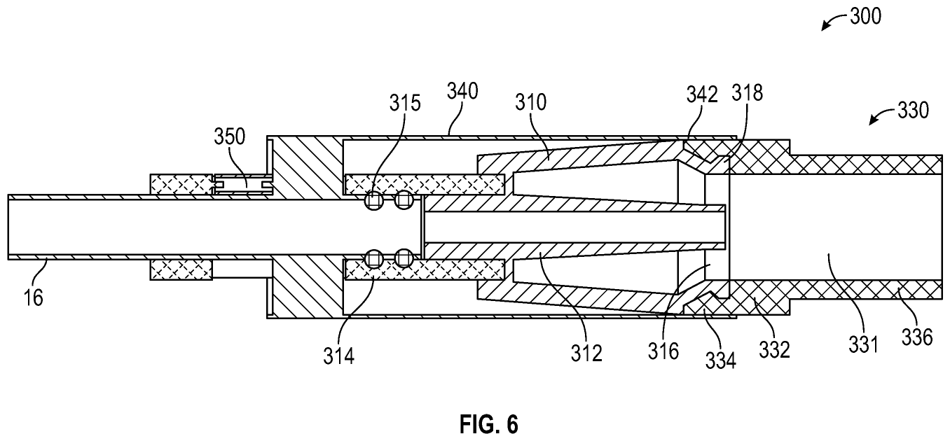

FIG. 6 illustrates a cross-sectional view of a fluid connector 300, in accordance with aspects of the present disclosure. With reference to FIG. 6, the fluid connector 300 provides a fluid pathway to a patient while allowing for selectable a “fusible link” or quick disconnect, allowing for a controlled disconnection of the fluid connector 300 if excess force is applied and allowing for a maintained connection during high pressure infusions or other procedures. As described herein, the fluid connector 300 can allow for controlled disconnection at a predetermined level of force to prevent a catheter from being inadvertently removed from the patient or prevent inadvertent disconnection during high pressure infusion procedures.

As illustrated, the fluid connector 300 provides a flow path or fluid pathway from one end of the fluid connector 300 to the opposite end of the fluid connector 300. As illustrated, the fluid connector 300 includes a first connector portion 330 and a second connector portion 310 that are coupled together to form a fluid pathway.

In the depicted example, the first connector portion 330 permits fluid flow to and from the patient or any other portion of the IV set. In the depicted example, the first connector portion 330 includes an end portion 336 to be connected to mating tubing or luer connector. As illustrated, a mating tubing or luer connector can be in fluid communication with the lumen 331. Fluid can pass to and from the fluid connector 300 through the mating luer connector via a lumen 331 defined in the first connector portion 330.

Further, the second connector portion 310 permits fluid flow to and from a fluid source or any other portion of the IV set. In the depicted example, the second connector portion 310 can be connected to a fluid source or other portion of the IV set. Tubing 16 or a connector can be received in an opening 314 of the connector housing and can be in fluid communication with a lumen 312 of the second connector portion 310.

In some embodiments, the tubing 16 can slide relative to the opening 314 of the connector housing while maintaining fluid communication with the opening 314 and/or the lumen 312. Optionally, the second connector portion 310 can include a seal 315 to maintain isolation between the lumen 312 of the connector housing and the slidable tubing 16. In some embodiments, the seal 315 can maintain a seal or isolation between the tubing 16 and the lumen 312 as the tubing 16 moves or slides relative to the connector housing.

During operation, the first connector portion 330 and the second connector portion 310 can be coupled together to permit flow between the first connector portion 330 and the second connector portion 310. In the depicted example, the lumen 331 of the first connector portion 330 can define a mating opening. Similarly, the second connector portion 310 can define a mating opening 316 in fluid connection with a housing volume, the lumen 312, and the tubing 16 or other IV set component coupled to the opening 314 of the second connector portion 310. As the first connector portion 330 and the second connector portion 310 are coupled together, fluid can flow between the mating opening of the first connector portion 330 and the mating opening 316 of the second connector portion 310, allowing flow between the first connector portion 330 and the second connector portion 310.

In the depicted example, the first connector portion 330 and the second connector portion 310 can be engaged to secure or retain the first connector portion 330 and the second connector portion 310 together. As illustrated, the first connector portion 330 and the second connector portion 310 can be coupled together by securing a portion of the first connector portion 330 around the second connector portion 310. In the depicted example, the first connector portion 330 includes or defines one or more engagement fingers 332. As illustrated, the engagement fingers 332 extends axially away from the first connector portion 330. Optionally, the engagement fingers 332 can be disposed circumferentially around the lumen 331 of the first connector portion 330. In some embodiments, the engagement fingers 332 can each define a ridge or lip 334 that extends radially inward toward the lumen 331. Similarly, the second connector portion 310 defines a groove 318. Optionally, the groove 318 can be disposed circumferentially around the lumen 312 of the second connector portion 310. Further the groove 318 can be defined on the outer surface of the second connector portion 310. During operation, the engagement fingers 332 of the first connector portion 330 can engage with the groove 318 of the second connector portion 310 to couple the first connector portion 330 and the second connector portion 310. In some embodiments, the lip 334 of the engagement fingers 332 can engage with the groove 318 of the second connector portion 310.

In the depicted example, the engagement fingers 332 and/or the lip 334 of the first connector portion 330 engages the groove 318 of the second connector portion 310 to resist movement of the first connector portion 320 and the second connector portion 310 in a direction away from each other. Engagement of the engagement fingers 332 against the groove 318 can, in some instances of the present disclosure, define a snap fitting or snap joint between the first connector portion 330 and the second connector portion 310. In some embodiments, the engagement fingers 332 can deflect to facilitate engagement or disengagement of the engagement fingers 332 with the groove 318.

Although the engagement fingers 332 are configured to resist separation of the first connector portion 330 and the second connector portion 310, the engagement fingers 332 and/or groove 318 are also configured to permit separation of the first connector portion 320 and the second connector portion 310 when a threshold force exceeded between the first connector portion 330 and the second connector portion 310. In some embodiments of the present disclosure, the threshold force for separating the first connector portion 330 and the second connector portion 310 is greater than or equal to approximately five pounds (22.25 Newtons). Separation of the first connector portion 330 and the second connector portion 310 can occur when the engagement fingers 332 of the first connector portion 330 are biased or flexed in a direction away from the groove 318. In the depicted example, the first connector portion 330 and the second connector portion 310 can separate from each other while maintaining the ability to reconnect the first connector portion 330 and the second connector portion 310 together. In some embodiments, the resistance or force to assembly and separation between the first connector portion 330 and the second connector portion 310 can be configured so that the force required for assembly of the first connector portion 330 and the second connector portion 310 is less than the force required for separation the first connector portion 330 and the second connector portion 310.

In some embodiments, the connector 300 can prevent the unintended disconnection of the first connector portion 330 and the second connector portion 310, during certain applications, such as high-pressure fluid infusions. In the depicted example, the second connector portion 310 can include a sliding portion 340 with a sliding cover 342 to selectively prevent the disengagement of the engagement fingers 332 of the first connector portion 330 from the groove 318 of the second connector portion 310.

In a locking position, the sliding cover 342 can be aligned with the engagement fingers 332 to prevent the radial or outward deflection of the engagement fingers 332, thereby preventing the disengagement of the engagement fingers 332 from the groove 318 of the second connector portion 310.

During operation, the sliding cover 342 can be moved or slid away from the engagement fingers 332 to allow the engagement fingers 332 to radially or outwardly deflect, permitting the engagement or disengagement of the first connector portion 330 from the second connector portion 310. A clinician may apply an external force to translate or move the sliding cover 342 to a desired position. As illustrated, the sliding cover 342 can be formed with the sliding portion 340. Optionally, the sliding portion 340 can be coupled to the tubing 16 to allow the sliding cover 342 and the tubing 16 to slide together. In some applications, force applied to the tubing 16 can be utilized to move the sliding cover 342 to an unlocked position. In some embodiments, the sliding cover 342 is circumferentially disposed around the second connector portion 310. In some embodiments, the second connector portion 310 can include a biasing member 350 to urge the sliding cover 342 toward a locked or retained position. Optionally, the biasing member 350 can be disposed between a base of the sliding portion 340 and a base portion of the second connector portion 310. In some applications, a clinician or patient (inadvertently) may apply an external force to the sliding cover 342 and/or the tubing 16 to overcome the spring force of the biasing member 350 to move the sliding cover 342 to an unlocked position.

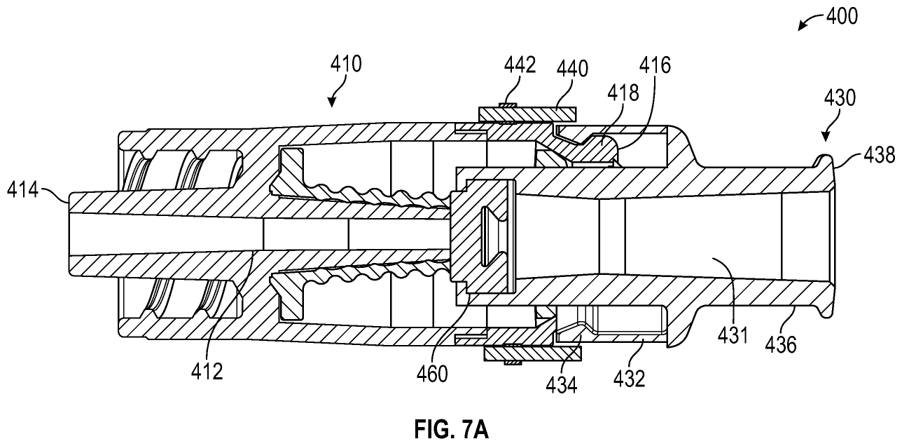

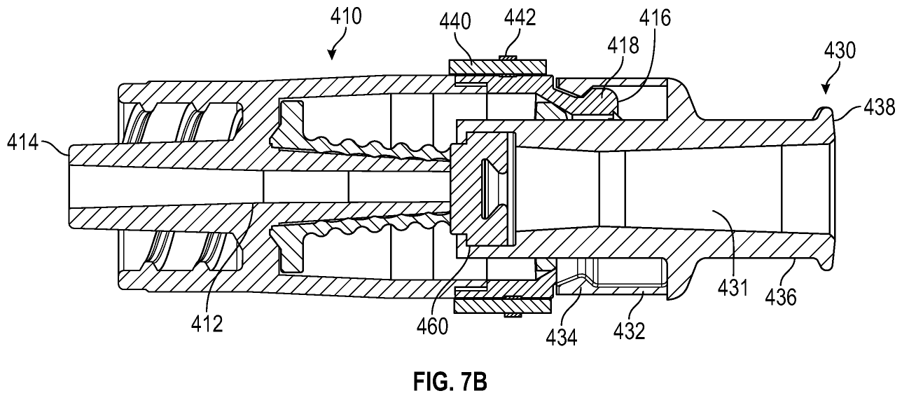

FIG. 7A illustrates a cross-sectional view of a fluid connector 400 in a locked configuration, in accordance with aspects of the present disclosure. FIG. 7B illustrates a cross-sectional view of a fluid connector 400 in an unlocked configuration, in accordance with aspects of the present disclosure. With reference to FIGS. 7A and 7B, the fluid connector 400 includes a locking ring 440. In the depicted example, the fluid connector 400 can include certain features that are similar to the features of fluid connector 300. Therefore, certain similar features may be referred to with similar reference numerals.

As illustrated, the fluid connector 400 provides a flow path or fluid pathway from one end of the fluid connector 400 to the opposite end of the fluid connector 400. As illustrated, the fluid connector 400 includes a first connector portion 430 and a second connector portion 410 that are coupled together to form a fluid pathway.

In the depicted example, the first connector portion 430 permits fluid flow to and from the patient or any other portion of the IV set. In the depicted example, the first connector portion 430 includes an end portion 436 to be connected to mating tubing or luer connector. In some applications, the end portion 436 defines a male luer portion 438. As illustrated, a mating tubing or luer connector can be in fluid communication with the lumen 431. Fluid can pass to and from the fluid connector 400 through the mating luer connector via a lumen 431 defined in the first connector portion 430.

Further, the second connector portion 410 permits fluid flow to and from a fluid source or any other portion of the IV set. In the depicted example, the second connector portion 410 can be connected to a fluid source or other portion of the IV set. Tubing or a connector can be received in an opening 414 of the connector housing and can be in fluid communication with a lumen 412 of the second connector portion 410.

During operation, the first connector portion 430 and the second connector portion 410 can be coupled together to permit flow between the first connector portion 430 and the second connector portion 410. In the depicted example, the lumen 431 of the first connector portion 430 can define a mating opening. Similarly, the second connector portion 410 can define a mating opening 416 in fluid connection with a housing volume, the lumen 412, and the tubing or other IV set component coupled to the opening 414 of the second connector portion 410. As the first connector portion 430 and the second connector portion 410 are coupled together, fluid can flow between the mating opening of the first connector portion 430 and the mating opening 416 of the second connector portion 410, allowing flow between the first connector portion 430 and the second connector portion 410.

In the depicted example, the first connector portion 430 and the second connector portion 410 can each include a valve member respectively to control flow through the connector 400. In particular, the valve member 460 allow for fluid to flow between the first connector portion 430 and the second connector 410 when the first connector portion 430 and the second connector portion 410 are connected and prevent fluid flow when the first connector portion 430 and the second connector portion 410 are disconnected.

Similar to the features described with respect to connector 300, the first connector portion 430 and the second connector portion 410 can be engaged to secure or retain the first connector portion 420 and the second connector portion 410 together. As illustrated, the first connector portion 430 and the second connector portion 410 can be coupled together by securing a portion of the first connector portion 220 around the second connector portion 210. In the depicted example, the first connector portion 430 includes or defines one or more engagement fingers 432 with an optional ridge or lip 234. Similar to connector 300, the second connector portion 410 defines a groove 418. In some embodiments, the engagement fingers 432 and/or the groove 418 of the connector 400 may function in a similar manner as the features of connector 300.

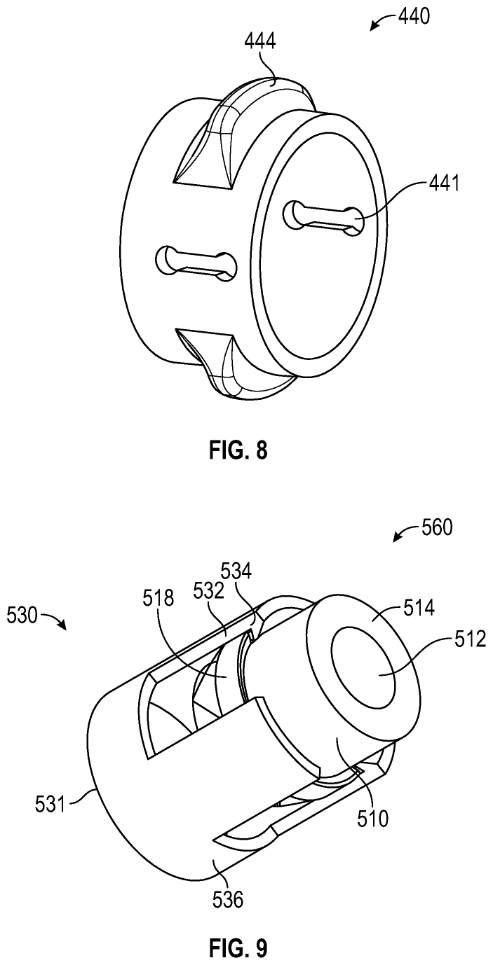

FIG. 8 illustrates a perspective view of a lock ring 440 of the fluid connector 400 of FIGS. 7A and 7B, in accordance with aspects of the present disclosure. With reference to FIGS. 7A-8, the connector 400 can utilize a lock ring 440 to prevent the unintended disconnection of the first connector portion 430 and the second connector portion 410, during certain applications, such as high pressure fluid infusions. In the depicted example, the second connector portion 410 can include a lock ring 440 to selectively prevent the disengagement of the engagement fingers 432 of the first connector portion 430 from the groove 418 of the second connector portion 410.

In a locking position (FIG. 7A), the lock ring 440 can be aligned with the engagement fingers 432 to prevent the radial or outward deflection of the engagement fingers 432, thereby preventing the disengagement of the engagement fingers 432 from the groove 418 of the second connector portion 410.

During operation, the lock ring 440 can be moved or slid away from the engagement fingers 432 to an unlocked position (FIG. 7B) to allow the engagement fingers 432 to radially or outwardly deflect, permitting the engagement or disengagement of the first connector portion 430 from the second connector portion 410. A clinician may apply an external force to translate or move the lock ring 440 to a desired position. In some embodiments, the lock ring 440 can include one or more protrusions 444 to allow a clinician to move or translate the lock ring 440.

In some embodiments, the lock ring 440 can be circumferentially disposed around the second connector portion 510. As illustrated, the lock ring 440 can be slidingly coupled to the second connector portion 410. In some embodiments, the lock ring 440 can be coupled to the outer surface of the second connector portion 410 via one or more snaps 442 extending from the second connector portion 410. The lock ring 440 can translate relative to the second connector portion 410 and the snaps 442 via one or more slots 441 defined in the lock ring 440. The slots 441 can define detents in the locked position and in the unlocked position to allow the lock ring 440 to be placed in retained in either the locked position or the unlocked position.

FIG. 9 illustrates a perspective view of a fluid connector 500, in accordance with aspects of the present disclosure. With reference to FIG. 9, the fluid connector 500 provides a fluid pathway to a patient while allowing for a selectable “fusible link” or quick disconnect, allowing for a controlled disconnection of the fluid connector 500 if excess force is applied and allowing for a maintained connection during high pressure infusions or other procedures. As described herein, the fluid connector 500 can allow for controlled disconnection at a predetermined level of force to prevent a catheter from being inadvertently removed from the patient or prevent inadvertent disconnection during high pressure infusion procedures. While certain elements of the fluid connector 500 are shown, other elements may be omitted for clarity. In some applications, the fluid connector 500 can include components depicted in other embodiments described herein.

As illustrated, the fluid connector 500 provides a flow path or fluid pathway from one end of the fluid connector 500 to the opposite end of the fluid connector 500. As illustrated, the fluid connector 500 includes a first connector portion 530 and a second connector portion 510 that are coupled together to form a fluid pathway.

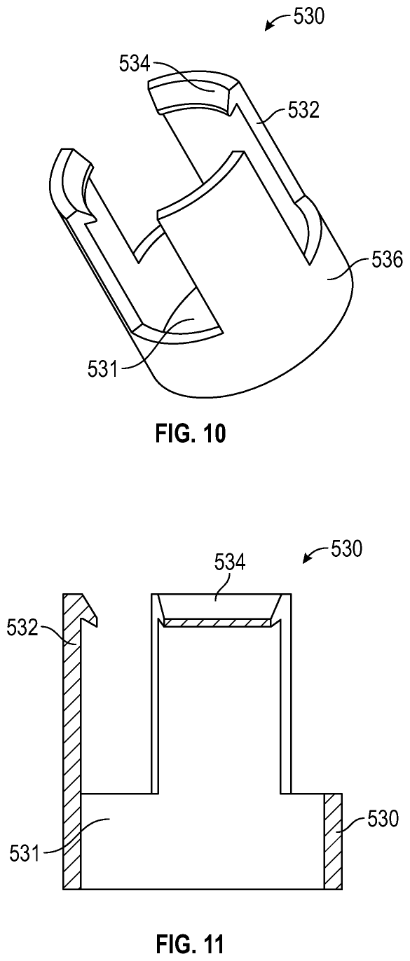

FIG. 10 illustrates a perspective view of a first connector portion 530, in accordance with aspects of the present disclosure. FIG. 11 illustrates a cross-sectional view of the first connector portion 530 of FIG. 10, in accordance with aspects of the present disclosure. With reference to FIGS. 9-11, the first connector portion 530 permits fluid flow to and from the patient or any other portion of the IV set. In the depicted example, the first connector portion 530 includes an end portion 536 to be connected to mating tubing or luer connector. As illustrated, a mating tubing or luer connector can be in fluid communication with the lumen 531. Fluid can pass to and from the fluid connector 500 through the mating luer connector via a lumen 531 defined in the first connector portion 530.

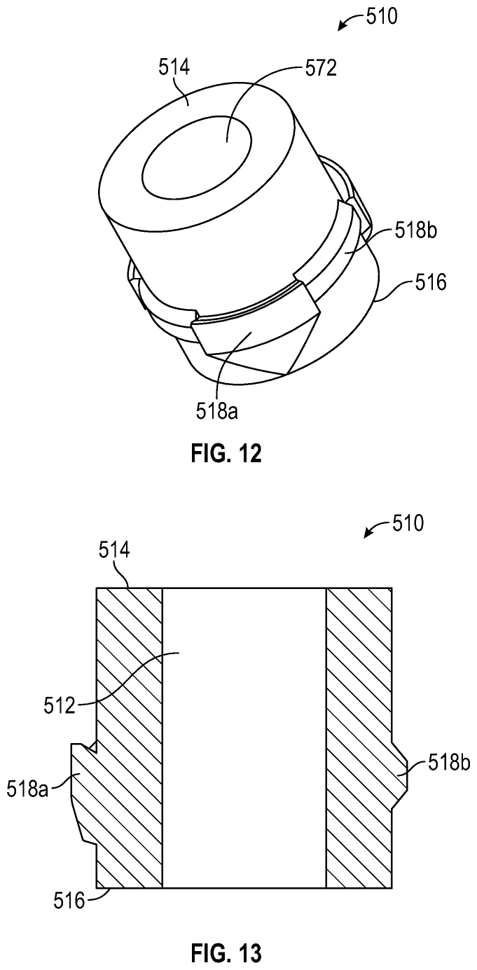

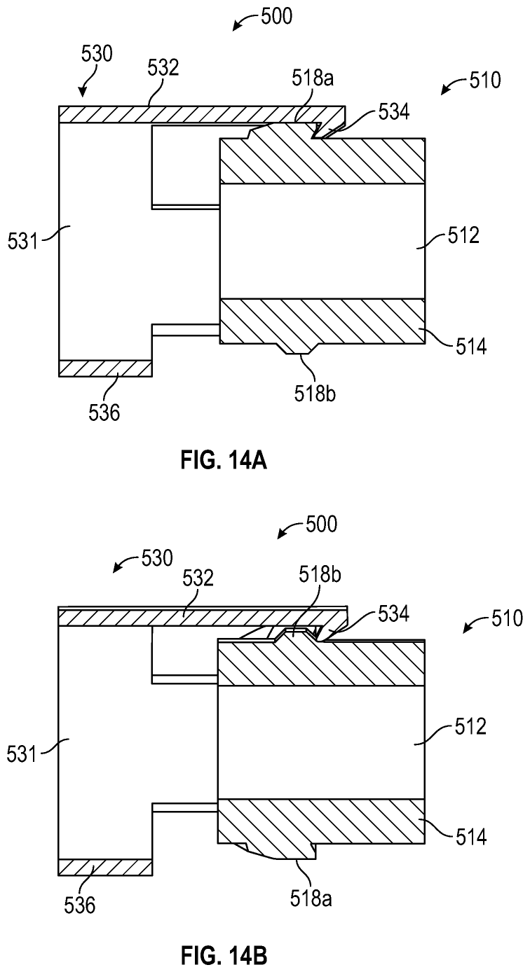

FIG. 12 illustrates a perspective view of a second connector portion 510, in accordance with aspects of the present disclosure. FIG. 13 illustrates a cross-sectional view of the second connector portion 510 of FIG. 12, in accordance with aspects of the present disclosure. With reference to FIGS. 12 and 13, the second connector portion 510 permits fluid flow to and from a fluid source or any other portion of the IV set. In the depicted example, the second connector portion 510 can be connected to a fluid source or other portion of the IV set. Tubing or a connector can be received in an opening 514 and can be in fluid communication with a lumen 512 of the second connector portion 510.

During operation, the first connector portion 530 and the second connector portion 510 can be coupled together to permit flow between the first connector portion 530 and the second connector portion 510. In the depicted example, the lumen 531 of the first connector portion 530 can define a mating opening. Similarly, the second connector portion 510 can define a mating opening 516 in fluid connection with a housing volume, the lumen 512, and the tubing or other IV set component coupled to the opening 514 of the second connector portion 510. As the first connector portion 530 and the second connector portion 510 are coupled together, fluid can flow between the mating opening of the first connector portion 530 and the mating opening 516 of the second connector portion 510, allowing flow between the first connector portion 530 and the second connector portion 510.

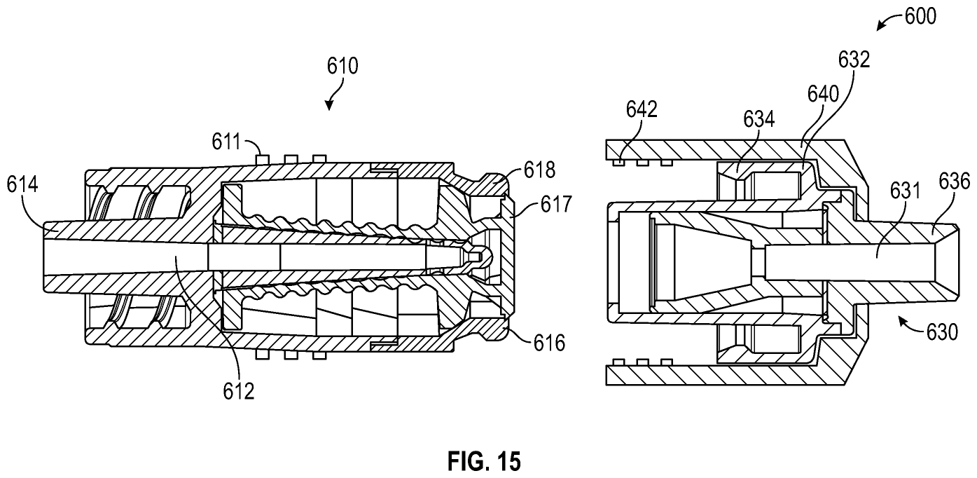

FIG. 14A illustrates a cross-sectional view of a fluid connector 500 in a locked configuration, in accordance with aspects of the present disclosure. With reference to FIG. 14A, the connector 500 can prevent the unintended disconnection of the first connector portion 530 and the second connector portion 510, during certain applications, such as high pressure fluid infusions. In the depicted example, the first connector portion 530 can include engagement fingers 532 that lock in engagement with one or more hooked lip portions 518a of the second connector portion 510.

In the depicted example, the first connector portion 530 includes or defines one or more engagement fingers 532. As illustrated, the engagement fingers 532 extends axially away from the first connector portion 530. Optionally, the engagement fingers 532 can be disposed circumferentially around the lumen 531 of the first connector portion 530. In some embodiments, the engagement fingers 532 can each define a hooked ridge or lip 534 that extends radially inward toward the lumen 531.

As illustrated, the second connector portion 510 defines one or more hooked lip portions 518a. Optionally, the hooked lip portions 518a can be disposed circumferentially around the lumen 512 of the second connector portion 510. Further the hooked lip portions 518a can be defined on the outer surface of the second connector portion 510. During operation, the engagement fingers 532 of the first connector portion 530 can engage with the hooked lip portions 518a of the second connector portion 510 to couple the first connector portion 530 and the second connector portion 510. In some embodiments, the lip 534 of the engagement fingers 532 can engage with the hooked lip portions 518a of the second connector portion 510.

In the depicted example, the engagement fingers 532 and/or the lip 534 of the first connector portion 530 engages the hooked lip portions 518a of the second connector portion 510 to resist movement of the first connector portion 530 and the second connector portion 510 in a direction away from each other. Engagement of the engagement fingers 532 against the hooked lip portions 518a can, in some instances of the present disclosure, define a snap fitting or snap joint between the first connector portion 530 and the second connector portion 510. In some embodiments, the engagement fingers 532 can deflect to facilitate engagement of the engagement fingers 532 with the hooked lip portions 518a.

In the locking position, the hooked lip portions 518a can engage with the hooked ridge or lip 534 of the engagement fingers 532 to prevent the radial or outward deflection of the engagement fingers 532, thereby preventing the disengagement of the engagement fingers 532 from the hooked lip portions 518a of the second connector portion 510.

FIG. 14B illustrates a cross-sectional view of a fluid connector 500 in an unlocked configuration, in accordance with aspects of the present disclosure. With reference to FIG. 14B, the connector 500 can selectively allow for disconnection to prevent damage to the connector and/or injury to a patient or clinician. In the depicted example, the engagement fingers 532 of the first connector portion 530 can be in releasable engagement with ramped lip portions 518b of the second connector portion 510. In some embodiments, the first connector portion 530 can be rotated to allow the engagement fingers 532 to be in locking engagement with the hooked lip portions 518a or in releasable engagement with the ramped lip portions 518b.

Similar to the hooked lip portions 518a, the second connector portion 510 defines one or more ramped lip portions 518b. Optionally, the ramped lip portions 518b can be disposed circumferentially around the lumen 512 of the second connector portion 510. Further the hooked lip portions 518a can be defined on the outer surface of the second connector portion 510. As illustrated, the ramped lip portions 518b can be disposed between the hooked lip portions 518a. During operation, the engagement fingers 532 of the first connector portion 530 can engage with the ramped lip portions 518b of the second connector portion 510 to releasably couple the first connector portion 530 and the second connector portion 510. In some embodiments, the lip 534 of the engagement fingers 532 can engage with the ramped lip portions 518b of the second connector portion 510.

In the depicted example, the engagement fingers 532 and/or the lip 534 of the first connector portion 530 engages the ramped lip portions 518b of the second connector portion 510 to resist movement of the first connector portion 530 and the second connector portion 510 in a direction away from each other. In some embodiments, the engagement fingers 532 can deflect to facilitate engagement and disengagement of the engagement fingers 532 with the ramped lip portions 518b.

Although the engagement fingers 532 are configured to resist separation of the first connector portion 530 and the second connector portion 510, in an unlocked position, the engagement fingers 532 and the ramped lip portions 518b are also configured to permit separation of the first connector portion 530 and the second connector portion 510 when a threshold force exceeded between the first connector portion 530 and the second connector portion 510. In some embodiments of the present disclosure, the threshold force for separating the first connector portion 530 and the second connector portion 510 is greater than or equal to approximately five pounds (22.25 Newtons). Separation of the first connector portion 530 and the second connector portion 510 can occur when the engagement fingers 532 of the first connector portion 530 are biased or flexed in a direction away from the ramped lip portions 518b. As illustrated, the ramp or wedge geometry of the ramped lip portions 518b can facilitate the radial expansion or outward deflection of the engagement fingers 532 to disengage the first connector portion 530 from the second connector portion 510. In the depicted example, the first connector portion 530 and the second connector portion 510 can separate from each other while maintaining the ability to reconnect the first connector portion 530 and the second connector portion 510 together. In some embodiments, the resistance or force to assembly and separation between the first connector portion 530 and the second connector portion 510 can be configured so that the force required for assembly of the first connector portion 530 and the second connector portion 510 is less than the force required for separation the first connector portion 530 and the second connector portion 510.

FIG. 15 illustrates a cross-sectional view of a separated fluid connector 600, in accordance with aspects of the present disclosure. With reference to FIG. 15, the fluid connector 600 can include a threaded cover 640 to prevent the unintended disconnection of the first connector portion 630 and the second connector portion 610. In the depicted example, the fluid connector 600 can include certain features that are similar to the features of fluid connector 400. Therefore, certain similar features may be referred to with similar reference numerals.

In the depicted example, the first connector portion 630 can include a threaded cover 640 to selectively prevent the disengagement of the engagement fingers 632 of the first connector portion 630 from the groove 618 of the second connector portion 610. In a locking position, the threaded cover 640 can be aligned with the engagement fingers 632 to prevent the radial or outward deflection of the engagement fingers 632, thereby preventing the disengagement of the engagement fingers 632 from the groove 618 of the second connector portion 610. In some applications, the threaded cover 640 can be secured or positioned in the locked position by engaging or screwing the threads 642 of the threaded cover 640 with threads 611 defined on an outer surface of the second connector portion 610.

During operation, the threaded cover 640 can be moved or slid away from the engagement fingers 632 to allow the engagement fingers 632 to radially or outwardly deflect, permitting the engagement or disengagement of the first connector portion 630 from the second connector portion 610. In some applications, the threaded cover 640 can be unscrewed or otherwise disengaged from the threads 611 defined on the second connector portion 610 to allow the threaded cover 640 to be moved away from the engagement fingers 632. In some embodiments, the threaded cover 640 is circumferentially disposed around the body of the first connector portion 630.

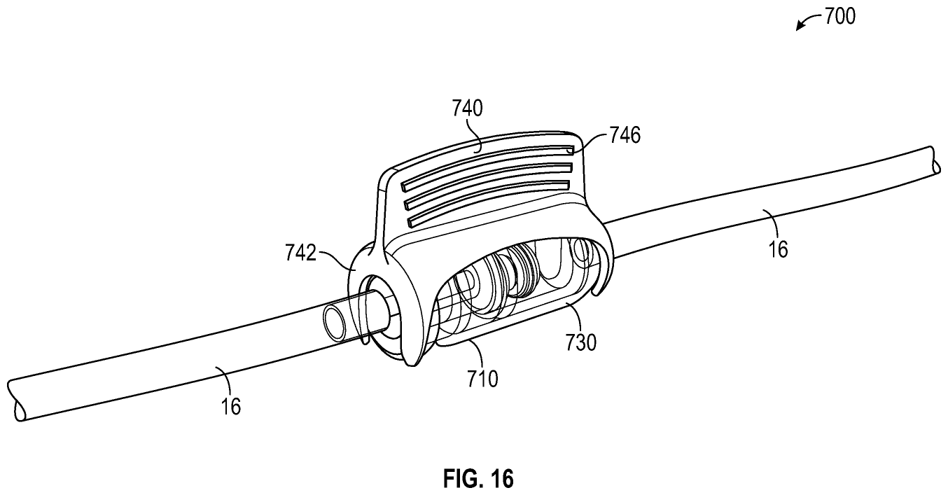

FIG. 16 illustrates a perspective view of a fluid connector 700 with a cover 740, in accordance with aspects of the present disclosure. With reference to FIG. 16, the fluid connector 700 includes removable cover 740 to provide a fluid pathway to a patient while allowing for a selectable “fusible link” or quick disconnect, allowing for a controlled disconnection of the fluid connector 700 if excess force is applied and allowing for the use of a removable lockout or cover 740 for a maintained connection during high pressure infusions or other procedures.

As illustrated, the fluid connector 700 provides a flow path or fluid pathway from one end of the fluid connector 700 to the opposite end of the fluid connector 700. As illustrated, the fluid connector 700 includes a first connector portion 730 and a second connector portion 710 that are coupled together to form a fluid pathway. In the depicted example, the first connector portion 730 permits fluid flow to and from the patient or any other portion of the IV set. In the depicted example, the first connector portion 730 can be connected to mating tubing 16 or a luer connector. Further, the second connector portion 710 permits fluid flow to and from a fluid source or any other portion of the IV set. In the depicted example, the second connector portion 710 can be connected to a fluid source or other portion of the IV set. Tubing 16 or a connector can be received in and can be in fluid communication with the second connector portion 710.

During operation, the first connector portion 730 and the second connector portion 710 can be coupled together to permit flow between the first connector portion 730 and the second connector portion 710. In the depicted example, the first connector portion 730 and the second connector portion 710 can be engaged to secure or retain the first connector portion 730 and the second connector portion 710 together.

Although the connector 700 is configured to resist separation of the first connector portion 730 and the second connector portion 710, the connector 700 is also configured to permit separation of the first connector portion 730 and the second connector portion 710 when a threshold force exceeded between the first connector portion 730 and the second connector portion 710.

In the depicted example, the connector 700 can include a removable cover 740 to selectively prevent the disengagement of the first connector portion 730 from the second connector portion 710. In an installed or locked position, the removable cover 740 can engage with the outer ends or edges of the first connector portion 730 and the second connector portion 710 to prevent the spacing apart or the disengagement thereof. As illustrated, arms 742 can extend from the cover 740 to engage with the ends of the first connector portion 730 and the second connector portion 710 while allowing tubing 16 to pass through the cover 740. Further, the body of the cover 740 may support the first connector portion 730 and the second connector portion 710 to remain in axial alignment. In some embodiments, the cover 740 can have an interference fit or a “snap fit” with the assembled connector 700 to maintain the positioning of the cover 740 relative to the first connector portion 730 and the second connector portion 710.

During operation, the cover 740 can be removed, permitting disengagement of the first connector portion 730 from the second connector portion 710. A clinician may apply removal force to a handle portion 746 of the cover 740 to remove the cover from the first connector portion 730 and the second connector portion 710.

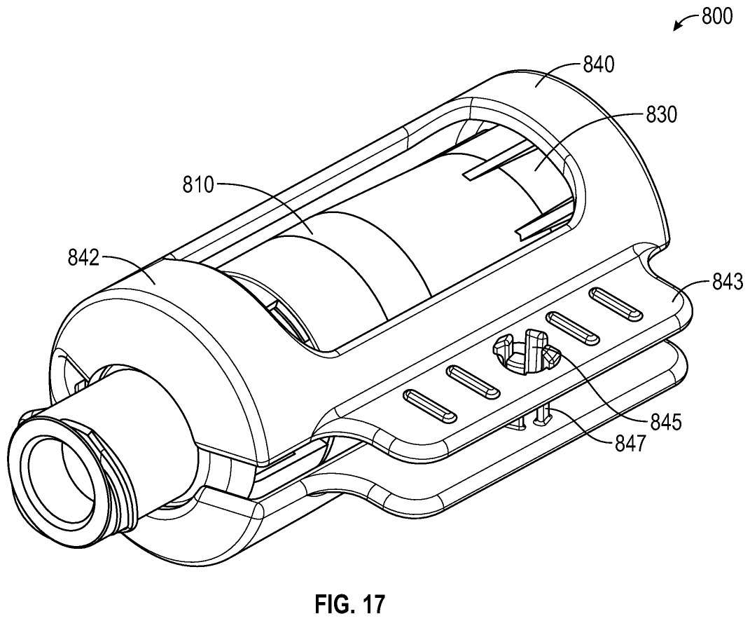

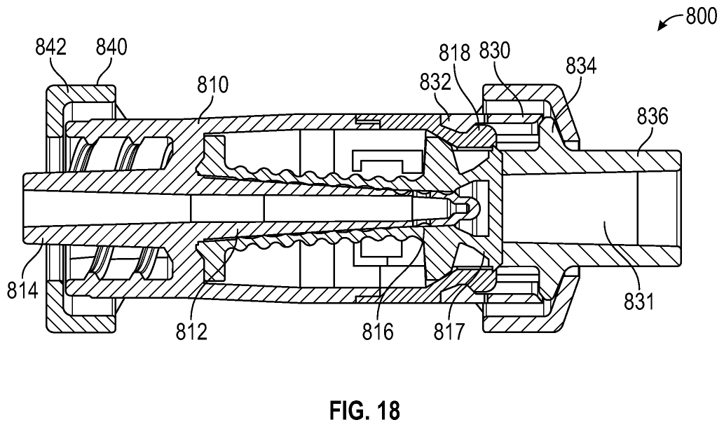

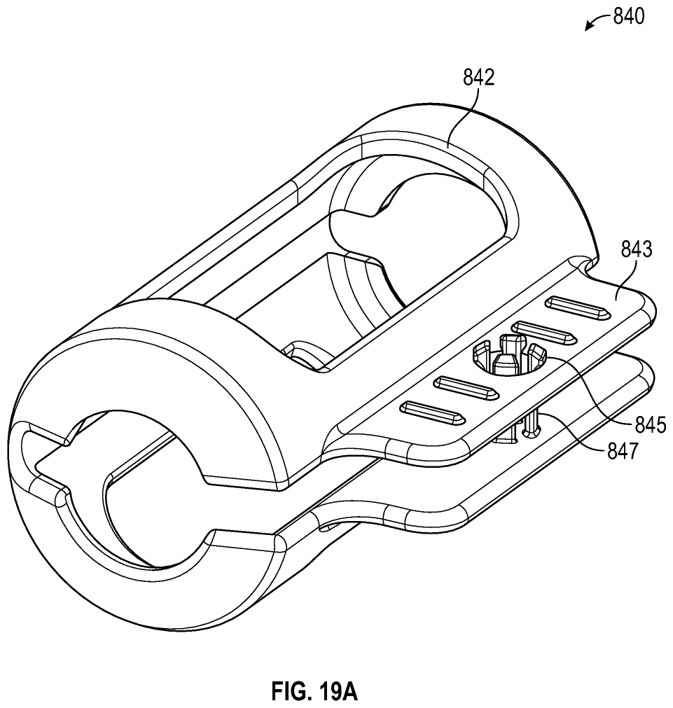

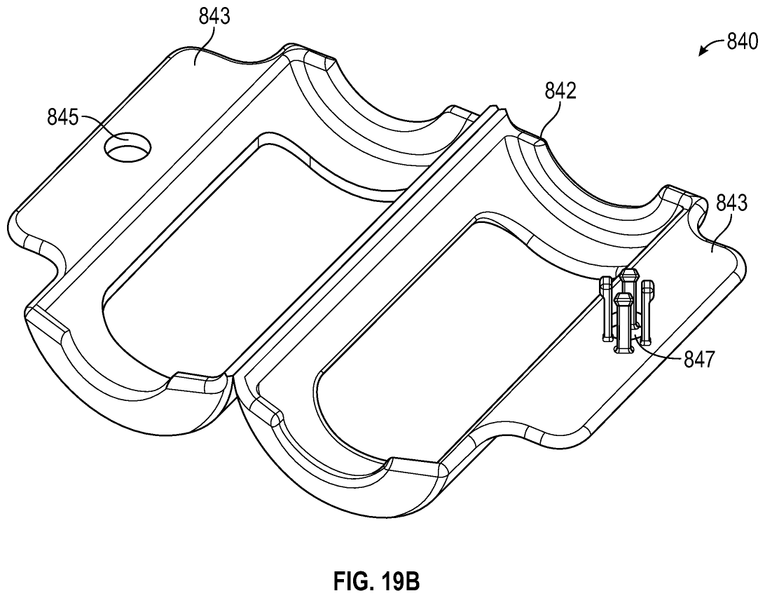

FIG. 17 illustrates a perspective view of a fluid connector 800 with a cover 840, in accordance with aspects of the present disclosure. FIG. 18 illustrates a cross-sectional view of a fluid connector 800 with a cover 840, in accordance with aspects of the present disclosure. FIG. 19A illustrates a perspective view of a cover 840 for a fluid connector 800 in a closed configuration, in accordance with aspects of the present disclosure. With reference to FIGS. 17-19A, the fluid connector 800 can include a hinged cover 840 to prevent the unintended disconnection of the first connector portion 830 and the second connector portion 810. In the depicted example, the fluid connector 800 can include certain features that are similar to the features of fluid connector 700. Therefore, certain similar features may be referred to with similar reference numerals.

In the depicted example, the connector 800 can include a removable cover 840 to selectively prevent the disengagement of the first connector portion 830 from the second connector portion 810. In an installed or locked position, the removable cover 840 can engage with the outer surfaces of the first connector portion 830 and the second connector portion 810 to prevent the spacing apart or the disengagement thereof. Further, the cover body 842 may support the first connector portion 830 and the second connector portion 810 to remain in axial alignment. As illustrated, the cover body 842 can be formed as an outer frame to surround the outer surfaces and/or edges of the first connector portion 830 and the second connector portion 810 while allowing a clinician to monitor fluid flow through the first connector portion 830 and the second connector portion 810. The cover body 842 can include openings to allow the cover 840 to engage with the ends of the first connector portion 830 and the second connector portion 810 while allowing tubing to pass through the cover 840. In some embodiments, the cover 840 can have an interference fit or a “snap fit” with the assembled connector 800 to maintain the positioning of the cover 840 relative to the first connector portion 7830 and the second connector portion 810.

FIG. 19B illustrates a perspective view of a cover 840 for a fluid connector 800 in an open configuration, in accordance with aspects of the present disclosure. With reference to FIG. 19B, during operation, the cover 840 can be removed from the connector 800, permitting disengagement of the first connector portion 830 from the second connector portion 810. A clinician may open the hinged cover body 842 to allow the first connector portion 830 and the second connector portion 810 to be removed from the cover 840. In some embodiments, the hinged cover body 842 includes two cover portions that are movable relative to each other about a hinge portion. Optionally, the hinge portion can be formed as a “living hinge” during a manufacturing process.

As illustrated, the hinged portions can be releasably fastened via one or more engagement fingers 847. Engagement fingers 847 can extend from one portion of the cover body 842 and engage with a mating portion of the cover body 842 to fasten the hinged portions of the cover body 842 and secure the cover 840 on the connector 800. The engagement fingers 847 can extend through an opening 845 defined in the opposite portion of the cover body 842 to retain the cover 840 in a closed position. A clinician can depress, compress, or otherwise deform the engagement fingers 847 to release the engagement fingers 847 from the opening 845 and open the cover body 842 to separate the hinged portions of the cover body 842 and remove the connector 800 from the cover 840.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A connector comprising: a first connector portion comprising: a connector housing comprising: a connector body defining a housing volume, a luer opening and a mating opening, wherein the housing volume is in fluid communication with the luer opening and the mating opening; and an engagement portion at least partially surrounding the connector body, the engagement portion defining an engagement lip extending radially toward the connector body, wherein the engagement lip is configured to releasably engage the connector housing with a mating connector portion; and a cover portion configured to selectively surround the engagement portion of the connector housing, wherein the cover portion is slidable to engage with the engagement portion of the connector housing and prevent release of the connector housing with the mating connector portion.

Clause 2. The connector of Clause 1, further comprising: a second connector portion comprising defining a second connector volume, a tubing opening and a second mating opening, wherein the second connector volume is in fluid communication with the tubing opening and the second mating opening, wherein the mating opening of the first connector portion and the second mating opening of the second connector portion are in fluid communication when the first connector portion and the second connector portion are in engagement, the first connector portion and the second connector portion are configured to be in separable engagement when the cover portion is spaced apart from the engagement portion of the connector housing and to be in locking engagement when the cover portion is engaged with the engagement portion of the connector housing.

Clause 3. The connector of Clause 2, wherein the second connector portion defines an engagement groove, and the engagement groove is configured to engage the engagement lip of the first connector portion.

Clause 4. The connector of Clause 1, wherein the first connector portion further comprises: a sliding portion defining a luer end and a housing end, the sliding portion defining a lumen extending between the luer end and the housing end, wherein the housing end of the sliding portion extends through the luer opening of the connector body to couple the sliding portion to the connector housing, lumen is in fluid communication with the housing volume, and the cover portion is coupled to the sliding portion.

Clause 5. The connector of Clause 4, wherein the first connector portion comprises a biasing member disposed between the connector housing and the cover portion and the biasing member is configured to urge the cover portion toward engagement with the engagement portion of the connector housing.

Clause 6. The connector of Clause 4, wherein the cover portion defines one or more protrusions extending radially toward the sliding portion, wherein the cover portion is slidable to engage the one or more protrusions with the engagement portion of the connector housing.

Clause 7. The connector of Clause 6, wherein the cover portion defines one or more slots between the one or more protrusions, and the cover portion is rotatable to space apart the one or more protrusions from the engagement portion of the connector housing and permit release of the connector housing with the mating connector portion.

Clause 8. The connector of Clause 4, comprising a sealing member disposed between the sliding portion and the connector body.

Clause 9. The connector of Clause 4, wherein the first connector portion comprises a valve member disposed in the mating opening, wherein the valve member prevents fluid flow when the first connector portion is spaced apart from the mating connector portion.

Clause 10. The connector of Clause 2, wherein the cover portion is coupled to the second connector portion.

Clause 11. The connector of Clause 10, wherein a tubing is in fluid communication with the second connector portion, the cover portion is coupled to the tubing, and the cover portion is movable with the tubing.

Clause 12. The connector of Clause 10, wherein the cover portion is movable to engage a first detent or a second detent, the cover portion being spaced apart from the engagement portion of the connector housing when engaging the first detent, and the cover portion being engaged with the engagement portion of the connector housing when engaging the second detent.

Clause 13. The connector of Clause 2, wherein the cover portion defines a threaded portion, the second connector portion defines a mating threaded portion, and the threaded portion of the cover portion is configured to threadedly engage the mating threaded portion of the second connector portion.

Clause 14. A connector comprising: a first connector portion defining a connector volume, a luer opening and a mating opening, wherein the connector volume is in fluid communication with the luer opening and the mating opening, the first connector portion defining an engagement lip extending radially from the first connector portion, wherein the engagement lip comprises a first lip portion configured to releasably engage an engagement finger of mating connector portion and a second lip portion configured to prevent release of the engagement finger of the mating connector portion.

Clause 15. The connector of Clause 14, further comprising: a second connector portion comprising defining a second connector volume, a tubing opening and a second mating opening. wherein the second connector volume is in fluid communication with the tubing opening and the second mating opening, the second connector portion defining one or more engagement fingers configured to engage the engagement lip of the first connector portion.

Clause 16. The connector of Clause 15, wherein the first connector portion is rotatable relative to the second connector portion between a first position and a second position, in the first position the one or more engagement fingers of the second connector portion engage the first lip portion of the engagement lip to allow for the first connector portion and the second connector portion to releasably engage, and in the second position the one or more engagement fingers of the second connector portion engage the second lip portion of the engagement to lip to prevent release of the first connector portion and the second connector portion.

Clause 17. The connector of Clause 14, wherein the first lip portion comprises a ramped portion.

Clause 18. The connector of Clause 14, wherein the second lip portion comprises a hooked portion.

Clause 19. A connector comprising: a first connector portion defining a connector volume, a luer opening and a mating opening, wherein the connector volume is in fluid communication with the luer opening and the mating opening; a second connector portion comprising defining a second connector volume, a tubing opening and a second mating opening. wherein the second connector volume is in fluid communication with the tubing opening and the second mating opening, wherein the first connector portion and the second connector portion are configured to be separably engageable and the mating opening of the first connector portion and the second mating opening of the second connector portion are in fluid communication when the first connector portion and the second connector portion are in engagement; and an attachable cover portion to selectively surround the first connector portion and the second connector portion and prevent release of the first connector portion and the second connector portion.

Clause 20. The connector of Clause 19, wherein the attachable cover portion comprises a first cover portion and a second cover portion coupled by a hinge.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A connector comprising:
a first connector portion comprising:
a connector housing comprising:
a connector body defining a housing volume, a luer opening and a mating opening, wherein the housing volume is in fluid communication with the luer opening and the mating opening; and
an engagement portion at least partially surrounding the connector body, the engagement portion defining an engagement lip extending radially toward the connector body, wherein the engagement lip is configured to releasably engage the connector housing with a mating connector portion; and
a cover portion configured to selectively surround the engagement portion of the connector housing, wherein the cover portion is slidable to engage with the engagement portion of the connector housing and prevent release of the connector housing with the mating connector portion.

2. The connector of claim 1, further comprising:
a second connector portion comprising defining a second connector volume, a tubing opening and a second mating opening, wherein the second connector volume is in fluid communication with the tubing opening and the second mating opening, wherein the mating opening of the first connector portion and the second mating opening of the second connector portion are in fluid communication when the first connector portion and the second connector portion are in engagement, the first connector portion and the second connector portion are configured to be in separable engagement when the cover portion is spaced apart from the engagement portion of the connector housing and to be in locking engagement when the cover portion is engaged with the engagement portion of the connector housing.

3. The connector of claim 2, wherein the second connector portion defines an engagement groove, and the engagement groove is configured to engage the engagement lip of the first connector portion.

4. The connector of claim 1, wherein the first connector portion further comprises:
a sliding portion defining a luer end and a housing end, the sliding portion defining a lumen extending between the luer end and the housing end, wherein the housing end of the sliding portion extends through the luer opening of the connector body to couple the sliding portion to the connector housing, lumen is in fluid communication with the housing volume, and the cover portion is coupled to the sliding portion.

5. The connector of claim 4, wherein the first connector portion comprises a biasing member disposed between the connector housing and the cover portion and the biasing member is configured to urge the cover portion toward engagement with the engagement portion of the connector housing.

6. The connector of claim 4, wherein the cover portion defines one or more protrusions extending radially toward the sliding portion, wherein the cover portion is slidable to engage the one or more protrusions with the engagement portion of the connector housing.

7. The connector of claim 6, wherein the cover portion defines one or more slots between the one or more protrusions, and the cover portion is rotatable to space apart the one or more protrusions from the engagement portion of the connector housing and permit release of the connector housing with the mating connector portion.

8. The connector of claim 4, comprising a sealing member disposed between the sliding portion and the connector body.

9. The connector of claim 4, wherein the first connector portion comprises a valve member disposed in the mating opening, wherein the valve member prevents fluid flow when the first connector portion is spaced apart from the mating connector portion.

10. The connector of claim 2, wherein the cover portion is coupled to the second connector portion.

11. The connector of claim 10, wherein a tubing is in fluid communication with the second connector portion, the cover portion is coupled to the tubing, and the cover portion is movable with the tubing.

12. The connector of claim 10, wherein the cover portion is movable to engage a first detent or a second detent, the cover portion being spaced apart from the engagement portion of the connector housing when engaging the first detent, and the cover portion being engaged with the engagement portion of the connector housing when engaging the second detent.

13. The connector of claim 2, wherein the cover portion defines a threaded portion, the second connector portion defines a mating threaded portion, and the threaded portion of the cover portion is configured to threadedly engage the mating threaded portion of the second connector portion.

14. A connector comprising:
a first connector portion defining a connector volume, a luer opening and a mating opening, wherein the connector volume is in fluid communication with the luer opening and the mating opening, the first connector portion defining an engagement lip extending radially from the first connector portion, wherein the engagement lip comprises a first lip portion configured to releasably engage an engagement finger of mating connector portion and a second lip portion configured to prevent release of the engagement finger of the mating connector portion.

15. The connector of claim 14, further comprising:
a second connector portion comprising defining a second connector volume, a tubing opening and a second mating opening, wherein the second connector volume is in fluid communication with the tubing opening and the second mating opening, the second connector portion defining one or more engagement fingers configured to engage the engagement lip of the first connector portion.

16. The connector of claim 15, wherein the first connector portion is rotatable relative to the second connector portion between a first position and a second position, in the first position the one or more engagement fingers of the second connector portion engage the first lip portion of the engagement lip to allow for the first connector portion and the second connector portion to releasably engage, and in the second position the one or more engagement fingers of the second connector portion engage the second lip portion of the engagement to lip to prevent release of the first connector portion and the second connector portion.

17. The connector of claim 14, wherein the first lip portion comprises a ramped portion.

18. The connector of claim 14, wherein the second lip portion comprises a hooked portion.

19. A connector comprising:
a first connector portion defining a connector volume, a luer opening and a mating opening, wherein the connector volume is in fluid communication with the luer opening and the mating opening;
a second connector portion comprising defining a second connector volume, a tubing opening and a second mating opening, wherein the second connector volume is in fluid communication with the tubing opening and the second mating opening, wherein the first connector portion and the second connector portion are configured to be separably engageable and the mating opening of the first connector portion and the second mating opening of the second connector portion are in fluid communication when the first connector portion and the second connector portion are in engagement; and
an attachable cover portion to selectively surround the first connector portion and the second connector portion and prevent release of the first connector portion and the second connector portion.

20. The connector of claim 19, wherein the attachable cover portion comprises a first cover portion and a second cover portion coupled by a hinge.
